(19) United States
(12) Patent Application Publication
(10) Pub. No.: US 2015/0322541 A1
(43) Pub. Date: Nov. 12, 2015

(54) A METHOD OF ADAPTATION OF A BACTERIAL CULTURE AND LEACHING PROCESS

(71) Applicant: BIOHEAP Limited, West Perth, WA (AU)

(72) Inventors: Jason Alexander FEWINGS, West Perth (AU); Shawn Ginn Ming SEET, West Perth (AU); Craig Geoffrey FITZMAURICE, Canning Vale (AU)

(73) Assignee: BIOHEAP Limited, West Pert, AU (AU)

(21) Appl. No.: 14/387,743

(22) PCT Filed: May 14, 2013
(86) PCT No.: PCT/AU2013/000496

§ 371 Date: Sep. 24, 2014

(30) Foreign Application Priority Data
May 14, 2012 (AU) 2012901968
Apr. 12, 2013 (AU) 2013204372

(51) Int. Cl. C22B 3/18; C22B 3/00; C12N 1/36

(52) U.S. Cl. CPC C22B 3/18; C12N 1/36; C22B 23/0407

(57) ABSTRACT

A method of adaptation of a bacterial culture, the method comprising the method steps of:
Exposing the bacterial culture to increasing levels of pH over a period of time.

A leaching process is also described.

### TECHNICAL FIELD

The present invention relates to a method of adaptation of a bacterial culture and leaching process. More particularly, the method of the present invention is particularly intended to facilitate the production of a bacterial culture for use in leaching under select conditions, including pH. Further, the leach of the present invention, utilising the adapted bacterial culture, is intended to provide reduced acid consumption and lower iron concentrations in the resulting pregnant leach solution when compared with biological leach processes of the prior art.

### BACKGROUND ART

The following discussion of the background art is intended to facilitate an understanding of the present invention only. The discussion is not an acknowledgement or admission that any of the material referred to is or was part of the common general knowledge as at the priority date of the application.

Biomining, biological leaching or bio-leaching is the utilisation of microorganisms to assist in the dissolution of valuable metals from ores. In general, the dissolution of mineral sulfides in bacterial systems is the result of ferric ion and proton attack via the polysulfide pathway (Rohwerder et al., 2008). The conditions for such a reaction are generally dictated by the ability of the microorganism to efficiently catalyze the leaching process.

Previous studies have demonstrated that bioleaching of mineral sulfides occurs favorably at pH<3 (Halinen et al., 2009), with the majority of heap bioleaching processes of low grade ore being treated with a solution pH between 1.5 to 2.5 (Plumb et al., 2008). At such pH conditions, the bio-oxidation acidophilic microorganisms convert ferrous ion to ferric ion which is needed for the leaching of sulfide minerals.

Bioleaching of materials at a high pH range has been studied previously (Cameron, et al., 2009a; Cameron, et al., 2009b; Cameron, et al., 2010; Halinen et al., 2009; Plumb et al., 2008). Importantly, the majority of results published suggest that above a pH of 2 the rate of leaching is significantly decreased and the percentage of metals recovered is reduced (Plumb et al., 2008). Halinen et al., 2009 reported the rate of bioleaching of nickel and zinc at pH 1.5 is 3 to 4 times faster than the rate of leaching observed at pH 3. It is suggested that the lack of dissolved ferric ion and diffusion barriers created by iron oxide precipitates minimised the rate of leaching at pH 2.5 to 3.

Interestingly, Cameron et al. (2009a; 2009b; 2010), has successfully demonstrated bioleaching of low-grade ultramafic nickel suphide ore at temperatures of 5° C. to 30° C., and 45° C., at pH 3 by mixed microbial culture previously adapted to the ore, using freshwater. The apparent success of Cameron's studies may be largely contributed to the adapting the microbes to specific conditions and feed materials prior to the bioleaching procedure.

The use of adapted microorganisms prior to leaching can enhance the leaching efficiency by 2 to 4 times when compared to unadapted culture (Li and Ke, 2001a; Li and Ke, 2001b).

Biomining is generally applied commercially as a heap leaching operation. A typical heap leach involves crushing and agglomeration of an ore or concentrate, followed by the stacking (for example 6-10 meters high) on an impermeable membrane. Sulfuric acid is percolated through the heap and additional aeration provided from at or near the bottom of the heap enhancing the microorganisms' growth. The native or inoculated microbial flora's growth causes the mineral dissolution, releasing the metal of interest in a leachate solution, or pregnant leach solution (PLS). The PLS is further processed for metal recovery by solvent extraction/electrowinning (SX/EW). The heterogeneous nature of the heap bio-leaching process results in large variation in temperature, particle size and reaction chemistry within the heap. Consequently, the rate of bio-leaching throughout the heap may vary, usually requiring extensive leaching periods of 300-450 days to ensure that as much of the heap is leaching effectively as possible.

One of the major costs associated with heap leaching operations is acid consumption. Acid consumption rates vary depending on the ore composition. This is further influenced by the operating pH and to a lesser extent the temperature.

Bioleaching of metal sulphides at extreme pH (<2) can result in the dissolution of gangue materials. Undesirably cations, such as aluminium, manganese, amorphous silica and specifically excessive ferric, can further result in releasing toxic trace elements, thickening of leach liquor thus potentially interfering with liquid flow in heap leaching, formation of passivation of sulphide minerals by jarosite formation or can be problematic during recovery of base metals during refinery (Dopso et al., 2009, Halinen et al., 2009).

Another important consideration for heap leaching is the downstream processing of the PLS. Final effluents from bioleaching operations generally have to be neutralised to remove iron and sulphate as stable end products by the addition of limestone or lime to increase of the pH of effluent to approximately 3. Therefore, it is understood by the Applicants that bioleaching of metals at high pH may potentially improve the downstream metals recovery processes, as well as reducing the operating costs.

The mineralogy of the specific ore can have a significant impact on the acid consumption properties and solution composition of the bacterial leaching system. Depending on the composition of the feed sample, the dissolution of mineral sulfides can be classified as either acid-producing or acid consuming. The majority of the gangue present in most ore samples, include magnesium silicates, and are acid consuming (Rawlings et al., 2003; Strömberg and Banwart, 1999). Therefore, maintaining of solution pH within a desirable range with the addition of sulfuric acid can be a major cost during a bioleaching operation (Watling, 2006). The application of pre-leaching of ore containing high levels of magnesium gangue prior to the bioleaching phase have shown to reduce time required to stabilize the pH level between 1.7-2.2 (Zhen et al., 2009; Qin et al., 2009). However, such a method has resulted in higher overall acid consumption at greater than 600 g kg-1 ore (Zhen et al., 2009; Qin et al., 2009). Halinen et. al. (2009) has demonstrated that an increase of pH from 1.5 to 2.0 during the column leaching of a particular black schist ore can reduce the acid consumption from 160 g kg-1 ore to 38 g kg-1 ore.

Ideally, in a heap leaching operation, target metals such as nickel and copper are solubilised with minimal acid consumption and iron dissolution. It is generally understood that microbial growth is favored at a lower pH. Accordingly, low pH, below about pH 2.5, has to date been utilised to increase leaching efficiency in heaps.

The present invention has as one object thereof to overcome substantially the abovementioned problems of the prior art, or to at least provide a useful alternative thereto.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The invention described herein may include one or more range of values (eg. size, displacement and field strength etc). A range of values will be understood to include all values within the range, including the values defining the range, and values adjacent to the range which lead to the same or substantially the same outcome as the values immediately adjacent to that value which defines the boundary to the range.

### SUMMARY OF INVENTION

In accordance with the present invention there is provided a method of adaptation of a bacterial culture for use in the treatment of ores and concentrates, the method comprising the method steps of:

Exposing the bacterial culture to increasing levels of pH over a period of time, whereby the bacterial culture is operative at a pH of above 2.5.

Preferably, the bacterial culture is operative at a pH of above 3.5.

Still preferably, the bacterial culture is operative at a pH of between about 3.5 to 5.

Preferably, the bacterial culture is a salt tolerant culture.

Still preferably, the salt tolerant culture is capable of operation at chloride concentrations of greater than about 100 g/L.

Still further preferably, the salt tolerant culture is capable of operation at total dissolved solids (TDS) levels of greater than about 115 g/L.

The temperature of step (i) is preferably about 45 to 55° C.

Still preferably, the temperature of step (i) is between about 50 to 55° C.

In one form of the present invention the bacterial culture is a bacterial culture capable of oxidising sulphide ores and concentrates.

In accordance with the present invention there is further provided a leaching process comprising the process steps of:
(i) Adapting a bacterial culture to operate at a pH of above 2.5; and
(ii) Leaching an ore or concentrate to which the adapted bacterial culture of step
(i) has been added or is added at a pH above 2.5.

Preferably, the adaptation of the bacterial culture of step (i) adapts that bacterial culture to operate at a pH above 3.5.

Still preferably, the adaptation of the bacterial culture of step (i) adapts that bacterial culture to operate at a pH between about 3.5 to 5.

Still further preferably, the adaptation of the bacterial culture of step (i) adapts that bacterial culture to operate at a pH of about 5.

The leaching of step (ii) is preferably conducted at the same or similar pH to that to which the bacterial culture has been adapted in step (i).

In one form of the present invention the bacterial culture may be adapted in step (i) to additional conditions, including conditions of salinity.

The leaching of step (ii) is preferably conducted at the same or similar additional conditions to that to which the bacterial culture has been adapted in step (i).

In one form of the present invention the leach is conducted in the form of a heap leach utilising one or more heaps. In another form of the present invention the leach is conducted in one or more tanks.

In a still further form of the present invention the leach provides a target metal recovery of greater than about 70% at a pH of between about 3.0 to 3.5. Preferably, the leach provides a target metal recovery of greater than 70% at a pH of greater than about 3.5. Still preferably, the target metal is nickel.

The leaching process of the present invention preferably consumes relatively low amounts of acid. Further, the leaching process of the present invention preferably produces a pregnant leach solution containing relatively low levels of iron.

### BRIEF DESCRIPTION OF THE DRAWINGS

The present invention will now be described, by way of example only, with reference to several embodiments thereof and the following drawings, in which:—

FIG. 1 is a plot of acid consumption of an ore sample crushed to P₁₀₀=9.5 mm and pH value of the solution maintained at a pH of 1.8;

FIG. 2 is a plot of the percentage of nickel recovered from the bioleaching of ore sample J062 containing high MgO using standard culture Ni—S-J065 and high pH adapted culture Ni—S-J069 under saline condition (100 g/L Cl⁻) at solution of 1.8, 2.8, 3 and 3.5;

FIG. 3 is a plot of redox potential (v Ag/AgCl) recorded during leaching of ore sample J062 containing high Mg) using culture N—S-J065 and high pH adapted culture Ni—S-J069 under saline condition (100 g/L Cl⁻) at solution pH 1.8, 2.8, 3 and 3.5;

FIGS. 4A and 4B are plots of total iron in solution from bioleaching of ore sample J062 containing high MgO using standard culture Ni—S-J065 and high pH adapted culture Ni—S-J069 under saline conditions (100 g/L Cl−) at solution pH of 1.8 (FIG. 4A) and pH 2.8, 3, 3.5 (FIG. 4B);

FIG. 5 is a plot of total cumulative quantity of acid added to each culture in order to maintain the pH at the respective value of 1.8, 2.8, 3 and 3.5;

FIG. 6 is a summary of the controls and variables utilised in the testing of Example 3;

FIG. 7 is a plot of residue corrected percentage of metal leached from amenability study on STR1, nickel ore with high Mg content maintained at pH 3.5 at a pulp density of 5% w/v.;

FIG. 8 is a plot of residue corrected percentage of metal leached from amenability study on STR2, nickel ore with high Mg content maintained at pH 3.5 at a pulp density of 5% w/v;

FIG. 9 is a plot of residue corrected percentage of metal leached from amenability study on STR3, nickel ore with high Mg content maintained at pH 3.5 at a pulp density of 5% w/v;

FIG. 10 is a plot of residue corrected percentage of metal leached from amenability study on STR4, copper concentrate maintained at pH 3.5 at a pulp density of 5% w/v;

FIG. 11 is a plot of metal residue corrected percentage of metal leached from amenability study on STR5, nickel arsenic tailings maintained at pH 2.0 at a pulp density of 5% w/v;

FIG. 12 is a plot of metal residue corrected percentage of metal leached from amenability study on STR6, nickel arsenic tailings maintained at pH 3.5 at a pulp density of 5% w/v. Due to iron precipitation at pH greater than 3 residue correction was not conducted for iron. Instead liquor assay result is used;

FIG. 13 is a plot of solution assay results of percentage of metal leached from amenability study on STR7, nickel ore with high Mg content maintained at pH 4 at a pulp density of 5% w/v. The test is completed but recovery results have not been adjusted with residue data due to delay in obtaining residues assay results;

FIG. 14 is a plot showing the results from monitoring of cumulative acid addition (), initial pH level (x), and adjusted pH levels (Δ) in STR1;

FIG. 15 is a plot showing the results from monitoring of cumulative acid addition (), initial pH level (x) and adjusted pH levels (Δ) pH levels in STR2;

FIG. 16 is a plot showing the results from monitoring of cumulative acid addition (), initial pH level (x) and adjusted pH levels (Δ) in STR3;

FIG. 17 is a plot showing the results from monitoring of cumulative acid addition (), initial pH level (x) and adjusted pH levels (Δ) in STR4;

FIG. 18 is a plot showing the results from monitoring of cumulative acid addition (), initial pH level (x) and adjusted pH levels (Δ) in STR5;

FIG. 19 is a plot showing the results from monitoring of cumulative acid addition (), initial pH level (x) and adjusted pH levels (Δ) in STR6;

FIG. 20 is a plot showing the results from monitoring of cumulative acid addition (), initial pH level (x) and adjusted pH levels (Δ) in STR7; and

FIG. 21 is a plot of the various proportions of bacterial species in the Applicant's high pH leaching bacterial culture.

### DESCRIPTION OF EMBODIMENTS

The method of adaptation of a bacterial culture of the present invention begins, in one form, with the selection of a salt tolerant bacterial culture. The selection of a salt tolerant bacterial culture is predicated on the observation that iron tends to precipitate from solution with high salt levels. Accordingly, salt tolerant bacterial cultures are adapted to leaching with low levels of iron in solution. The pH of the bacterial culture is increased in pH increments of 0.5 to a pH of at least 2.5, for example 3.5, at which point the majority of iron is precipitated. The bacterial culture of the present invention is adapted to operate effectively at 50 to 55° C. However, a range of about 45 to 55° C. is anticipated by the Applicants. The Applicant has deposited a sample of such an adapted bacterial culture, specifically prepared for use in the leaching of base metals, with the Australian Government's National Measurement Institute as Accession No. V08/027581. Additional samples specifically prepared for use in the leaching of copper and nickel have also been deposited with Accession Nos. V08/027580 and V08/027582, respectively.

The outcome of the adaptation process is assessed and if considered successful the pH is increased further in smaller increments. Kinetic and recovery data of the culture is noted as a measure of performance.

The tests of the bacterial culture are conducted in agitated vessels as opposed to shakers so as to mimic as closely as possible industrial conditions. The culture undergoing adaptation is split and further adapted, or re-adapted, to conditions of other starter cultures. Such conditions may include fresh water. The starter cultures are then mixed with the adapted culture and the process repeated. This is intended to produce pH resistance, or adaptation of high pH, in each of a range of cultures, being for example copper fresh, copper salt, base metal fresh, base metal salt, high arsenic, ferric oxidisers and so on.

It is envisaged that a final pH target of 10 may be achievable to facilitate gold leaching.

The adapted bacterial culture of the present invention may be utilised in a leach process comprising the process steps of:

Adapting a bacterial culture to operate at a pH of above 2.5; and

Leaching an ore or concentrate to which the adapted bacterial culture of step (i) has been added or is added in one or more heaps at a pH above 2.5.

The adaptation of the bacterial culture of step (i) adapts that bacterial culture to operate at a pH above 3.5, for example between about 3.5 to 5, further at about 5.

The leaching of step (ii) is conducted at the same or similar pH to that to which the bacterial culture has been adapted in step (i). The bacterial culture may be adapted in step (i) to additional conditions, including conditions of salinity.

The leaching of step (ii) is conducted at the same or similar additional conditions to that to which the bacterial culture has been adapted in step (i).

The leaching process of the present invention is envisaged to consume relatively low amounts of acid. Further, the leaching process of the present invention produces a pregnant leach solution containing relatively low levels of iron.

Iron is precipitated in one or more of the or each heap, or the or each leach tank, dependent upon the form of leach utilised. It is envisaged that the leaching process of the present invention will either not require a precipitation circuit or the precipitation circuit required will be significantly smaller than that required by prior art operations of a similar size and nature.

The present invention will now be described with reference to several non-limiting examples, as follows.

### Example 1

The objective of this initial test work was to develop a proprietary bacterial culture of the Applicant, being termed culture Ni—S-J065, so that the culture is capable of operating at a higher pH of 2.5, 3.0 or 3.5. The culture Ni—S-J065 is the culture referred to above and deposited under Accession No. V08/027581. The development of the culture to operate at higher pH was be carried out in a stepwise procedure, under saline conditions of 100 g/L or (115 g/L TDS) and at 50-55° C.

Each stage of the development ran for 3 months, while targeting >70% Ni oxidation before each scale up. Bacterial counts will also be monitored for growth and development.

Culture Development—Batch stirred tank reactor tests to develop culture Ni—S-J065 to leaching metal from a first ore sample at high pH.

Amenability Study—The best performing culture will be selected to be used in an amenability and compare the performance of the high pH leaching culture to the original Ni—S-J065 culture.

### 1. General Procedures

1.1. OK Nutrient Media (pH 1.6-1.8)

| Ingredient | Quantity | Unit |
|---|---|---|
| (NH₄)₂SO₄ | 1 | g |
| K₂HPO₄ | 0.5 | g |
| MgSO₄•7H₂O | 0.16 | G |
| Tap Water or Distilled | 1 | L |
| Water to Make | | |
| volume to: | | |

Dissolve all solids in 500 mL of distilled or tap water, pH adjust to 1.6-1.8 with H2S04 (98% AR grade), before makeup up to 1 L with distilled or tap water.

Solution Sample Preparation for Assay

1.1.1. Sampling
I. The sides of the test reactor should be washed down with water to ensure that all sulphides are available for oxidation, any evaporative losses should be made up with water.
II. The pH, ORP and dissolved oxygen content of the reactor test should be determined by inserting the pH, ORP and DO probe into the stirred aerated test.
III. Measure and record the pH, adjust if necessary, with the addition of 98% sulphuric acid. Record any additions, pH, ORP and DO readings in the daily data sheet.
IV. After any acid addition remeasure the pH₂ORP and DO and record on the daily data sheet
V. Ensure calibration of the dilution equipment has been performed. See “Procedure for Dilution Calibration”.

1.2.2. Solution Preparation
I. Via the plastic pipette remove approximately 8 ml of slurry and filter. Each vat is to have its own plastic pipette to avoid contamination.
II. Using the calibrated pipette take a 6 ml aliquot of solution and place in a clean vial/test tube
III. To this aliquot of solution add 3 ml of HCl (The acid addition will stabilise the solution and prevent any precipitates forming if the sample is not assayed immediately).
IV. Return the solids from the filter paper back into the reactor, and any excess solution not used for assay or ferrous titrations.

Calibration of Diluent (use for preparation for solution assay)

1.1.2. Determining SG of HCl.
I. Using a 10 ml glass volumetric pipette, get the weight of 10 ml of HCl
II. Calculate the SG of the HCl (weight/volume=SG)
III. Record on the calibration sheet
IV. The bottle of acid states SG as 1.16

1.1.3. Calibration of Bottle Top Dispenser.
I. Weigh (tare off balance) a solution vial to four decimal points.
II. Dispense a 3 ml aliquot of HLC into vial.
III. Weigh vial and aliquot.
IV. Record the weight of the aliquot on the calibration sheet.
V. The weights should be between 3.46-3.50 g. Ideal weight is 3.48 g.
VI. If the recorded weights are not within range the bottle top dispenser needs to be adjusted and calibration re-performed.
VII. Record the weight in the calibration sheet.
VIII. Calibration sheet will calculate the volume of HCl (weight of HCl/SG HCl).
IX. Repeat 3 times, recording each value.
X. The calibration sheet will calculate the average volume.

1.1.4. Calibration of Pipette.
I. Weight (tare off balance) a solution vial to four decimal points.
II. Dispense a 6 ml aliquot of DI water into vial.
III. Weigh container and solution.
IV. Record weight of aliquot in calibration sheet.
V. The weight should be between 5.98-6.02 g. Perfect weight is 6.00 g.
VI. If the recorded weights are not within range the pipette needs to be adjusted and calibration re-performed.
VII. Repeat 3 times, recording each value.
VIII. The calibration sheet will calculate the average volume.

Check calculation sheet to ensure averaged dilution factor is between 1.49 and 1.51.

Ferrous Iron Titration

1.1.5. Titration procedures
I. Calibrate the pipette to 1 ml using DI water.
II. Place a 1 mL aliquot of sample into a small conical flask.
III. Add 25 mL of buffer solution and a few drops (3) of iron indicator solution.
IV. Titrate with standard potassium dichromate solution from green to purple end point.
V. Ensure correct mixing is used and allow a few seconds between additions to ensure reaction is complete, on occasion the sample will immediately turn purple but after a few moments return to clear or green. More potassium dichromate needs to be added if this occurs.

1.1.6. Titration Calculation
Reaction

Cr₂O₇²⁻+14H⁺+6Fe²⁺→2Cr³⁺+7H₂O+6Fe³⁺
Calculation if using Potassium Dichromate Stock solution (0.49 g/1000 ml)

Fe²⁺ ppm=mL of titration×557.6

1.1.7. Titration Solution

1.1.7.1. Buffer Solution

| Ingredient | Quantity | Unit |
|---|---|---|
| H₃PO₄ (85%) | 300 | mL |
| H₂SO₄ (98%) | 300 | mL |
| Deionised Water | 1.4 | L |

1.1.7.2. Iron Indicator

| Ingredient | Quantity | Unit |
|---|---|---|
| Sodium | 0.5 | g |
| Diphenylamine | | |
| Sulphate | | |
| H₂SO₄ (98%) | 100 | mL |
| Deionised Water | 1.4 | L |

1.1.7.3. Standard Potassium Dichromate Stock Solution
I. Dry K₂Cr₂O₇ at 105° C. for 2 hours. Keep this chemical in a moisture free cupboard or desiccator.

| Ingredient | Quantity | Unit |
|---|---|---|
| Dry K₂Cr₂O₇ | 0.49 | g |
| Deionised Water | 1 | L |

### Other Procedures:

Sample Preparation
2.1.1. Find 2 kg of ore Samples. Upon receipt of the ore sample(s) the ore will be weighed and logged into the laboratory. The following needs to be carried out
   I. The sub-sample to be dried at a low temperature to determine the percent moisture.
   II. 2.5 kg of ore should be split using rotary splitter to provide samples as feed for bacterial development work and amenability study.
2.1.2. Submit samples for the subsequent:
   I. Head assay (0.5 kg) for the following: Ni, Co, Zn, Cu, Fe, Al, Ca, Mg, Mn, S (Total), S (Elemental), S²⁻ (Sulphide), SO₄²⁻, C (Total), CO₃²⁻, and an ICP Scan.
   II. Grind 2 kg of split ore to P80<75 um to be used as feed.

Saline Adaptation
2.1.3. A single bacterial culture development will be carried out on a first sample ground to 80% passing 75 um. All test work will be carried out in 115 g/L TDS using sample saline site water. The amount of salt to be added per litre of solution is:
   1 g of salt in 1 L of solution=1.16 g/L TDS Sample Site Saline water is ˜50 g/L TDS.
   Method 1:

$$Differences in salt concentration factor = 115 / 50 = 2.3$$

Needs to increase the salt level by 2.3 times.
50 g/L TDS÷1.16 g/L TDS=43.103 g/L pool salt
43.103 g/L×2.3=99.14 g/L pool salt
99.14 g/L−43.103 g/L=56.03 g/L pool salt to be added
Method 2:

$$Differences in salt concentration factor = 115 / 50 = 2.3$$

Needs to increase the salt level by 2.3 times.
50 g/L TDS÷1.16 g/L TDS=43.103 g/L pool salt.
115 g/L TDS÷1.16 g/L TDS=99.14 g/L pool salt
99.14 g/L−43.103 g/L=56.04 g/L pool salt to be added
Method 3:

$$Differences in salt concentration = 115 - 50 = 65 g \text{/} L TDS$$

65 g/L TDS÷1.16 g/L TDS=56.03 g/L pool salt to be added
Conclusion:
For every 1 L of Sample Saline Site water, add 56.03 g of pool salt.

Summary of Salt information

| Description | | Units |
|---|---|---|
| Based on previous assay 1 g/L of pool salt | = | 1.16 g/L of salt in TDS |
| TDS in Bacteria Stock J065 and SQ | = | 50.00 g/L of TDS |
| bacterial | | |
| Equivalent of pool salt in 50 g/L TDS | = | 43.10 g/L of pool salt |
| Equivalent of pool salt in 50 g/L TDS in 3 L | = | 129.31 g of pool salt in 3 L |
| Equivalent of pool salt in targeted 115 g/L | = | 99.14 g/L of pool salt |
| TDS | | |
| Equivalent of pool salt in targeted 115 g/L | = | 297.41 g of pool salt in 3 L |
| TDS in 3 L | | |
| Amount of pool salt needed to add to bring | = | 56.03 g/L of pool salt |
| 43.10 g/L to 133.40 g/L. | | |
| Amount of pool salt needed to add to bring | = | 168.10 g of pool salt in 3 L |
| 43.10 g/L to 133.40 g/L in 3 L solution. | | |
| Amount salt added per 5 days | = | 33.62 g/scaling up day |
| Amount of salt added per ½ day | = | 16.81 g/half day |

i. Prior to the start of the test work J065 culture needs to be grown for approximately a week before starting on Bacterial development test work. The reason for this is the need to increase the salt level gradually for the work. Gradual conditioning of salt level is undertaken in accordance with the Table immediately below:
   Salt Addition Schedule

| | | | | Final TDS |
|---|---|---|---|---|
| | Salt Concentration (g) | | | of pool |
| | | Salt | | Salt |
| Day | Starting | added | Final | (g/L) |
| Day 1 | 129.31 | 33.62 | 162.93 | 63 |
| Day 2 | 162.93 | 33.62 | 196.55 | 76 |
| Day 6 | 196.55 | 50.43 | 246.98 | 95.5 |
| Day 8 | 246.98 | 50.43 | 297.41 | 115 |

II. To start the adapting Ni—S-J065 culture to high salt levels first make up 2.5 L of OK solution using site water, follow by the addition of 33.62 g of pool salt.
III. Weigh a clean reactor and record weight.
IV. Add 500 mL volume of Ni—S-J065A bacteria to the reactor, weigh and record on sheet. Make up final volume to 3 L with the 2.5 L of saline Ok solution as prepared in step (I), then weigh reactor+content and record.
V. Place an accurate level mark on the side of the reactor for daily solution evaporation make up.
VI. Record pH (adjust if necessary pH to 2.0 with 98% H₂S0₄), ORP and Temp
VII. Add 30 g concentrate to 3 L of bacteria (5% w/v) and Ok solution as prepared in step II.
IX. Place the reactor into the 50-55° C. water bath.
IX. Allow the slurry to mix for 30 minutes then measure pH, ORP and DO and adjust pH with 98% H₂S0₄ if necessary.
X. Evaporation make up should be done on a daily basis using distilled water, prior to any measurements.
XI. Daily measure the pH, ORP and DO is measured. The pH adjustment is done with 98% H₂S0₄ and all additions recorded, (pH is to be adjusted before ORP measurement is taken).
XII. Bacteria numbers to be monitored every 2 days.
XIII. Salt to be added according to the salt addition schedule (see above).
XIV. Once bacteria numbers are up and bacteria are performing well 500 mL of the culture shall be used for the study. The remaining culture shall be stored and labeled Ni—S-J069A-01.

Bacterial Development
XV. Make up 2.5 L of OK solution using Sample Saline Site water and add 140.075 g of salt.
XVI. Weigh a clean reactor and record weight.
XVII. Add 500 mL volume of the Ni—S-J069A-01 bacteria (that has been conditioned to high salinity of 115 g/L TDS) to the reactor, weigh and record on sheet. Make up final volume to 2.5 L with Ok solution as prepared in section 2.3 step (I) then weigh reactor+content and record.
XVIII. Place an accurate level mark on the side of the reactor for daily solution evaporation make up.
XIX. Record pH (adjust if necessary pH to 2.0 with 98% H₂S0₄), ORP and Temp
XX. Remove a sample for analysis as per “Procedure for Preparing Solution Samples for Analysis”. Ensure that the solids are returned to reactor after filtering
XXI. Per test sample add 150 g of ground feed solid to 3 L of bacteria (5% w/v) and Ok solution as prepared in step II.
XXII. Place the reactor into the 50-55° C. water bath.
XXIII. Allow the slurry to mix for 30 minutes then measure pH, ORP and DO and adjust pH with 98% H₂S0₄ if necessary.
XXIV. Remove a second sample for analysis as per “Procedure for Preparing Solution Samples for Analysis”. Ensure that all the solids are returned to reactor after filtering.
XXV. Perform dilutions and titrations on both samples, any remaining filtrate to be returned to the reactor. Diluted solution to be submitted for assaying of the appropriate metals in solution.
XXVI. Evaporation make up should be done on a daily basis using distilled water, prior to any measurements and sample removal.
XXVII. Daily the pH, ORP and DO is measured. The pH adjustment is done with 98% H₂S0₄ and all additions recorded, (pH is to be adjusted before ORP measurement is taken).
XXVIII. A slurry sample is to be removed and filtered, on the samples designated days, for assaying of Ni, Co and Fe reporting to solution. Solution assay shall be conducted 3 times a week for the first 2 weeks, followed by once a week for 2.5 months. The bacterial numbers should be monitored once a week.
XXIX. Once Ni reporting to solution has reached >70% split the culture into halves. Store one half and rescale the other half using step wise procedure of half and feed stock culture. When scaling up the culture added 84.045 of salt to 1.5 L of Ok solution (use Sample Saline Site Water) before adding to the stock. If in sufficient water was added to bring the volume to the 3 L mark line, add the required volume of Sample Saline Site water. This time increase pH to 2.5.
XXX. Continue to repeat procedure as listed in Section 2.3 from point III to XV and increase pH by 0.5 every time the culture is halved and re-scaled, until pH value 3.5 is reached.

Adaptation and Amenability
2.1.4. Two amenability tests are conducted on the first ore sample using the Applicant's cultures, Ni—S-J069A-01 and the best culture developed from section 2.3. A volume of 500 mL of culture Ni—S-J069A-01 is restarted without adaption using procedure as listed in section 2.3 [added 140.075 g of salt to 2.5 L OK media (using saline water to make it up) to bring the vat volume to 3 L]. The newly developed culture will be restarted following half and feed procedure [added 840.045 g of salt to 1.5 L OK media (using saline water to make it up) to bring the vat volume to 3 L]. To each culture 300 g of ore (10% w/v) will be added. The pH of Ni—S-J069A-01 will be maintained at 1.8 and the newly developed culture will be kept at value that the culture was developed from in section 2.2.
   I. Evaporation make up should be done on a daily basis using distilled water, prior to any measurements and sample removal.
   II. Daily the pH, ORP and DO is measured as set out in section 2.2.1. The pH adjustment is done with 98% H₂S0₄ and all additions recorded, (pH is to be adjusted before ORP measurement is taken).
   III. A slurry sample is to be removed and filtered, on the samples designated days, for assaying of Ni, Co, and Fe reporting to solution. Solution assay shall be conducted 3 times a week.
   IV. Staff will on occasions ask for a SG to be performed on a sample. Filter approximately 12 ml, using a 10 ml pipette, record the weight of solution to four decimal places.
   V. Once the metal level is close to 100% oxidation the adaptation phase of the test is completed and the amenability test can be terminated.

Termination of Amenability Study
2.1.5. Once metal reporting in solution has plateaued, amenability study is completed. The procedure for terminating amenability study is as follows:
   I. Ensure daily top up and measurements have been taken and recorded for the test.
   II. Turn off overhead stirrer and air supply.
   III. Remove vat from water bath (use some paper towel to dry outside of vat).
   IV. Weigh vat and record on Final STR Data Summary sheet.
   V. Measure the volume of slurry contained within the vat.
   VI. Add floc (E10) to slurry record amount on sheet (not always required, only if sample has shown slow filtering characteristics during the test).
   VII. Filter using a vacuum flask and funnel.
   VIII. Measure the volume of the filtered final solution (filtrate) and take an aliquot of the filtered final solution for assay.
   IX. Record the total weight of the filtrate. This can be done by recording the tare of the collection vessels and then the total weight of solution plus the collection vessel(s).
   X. Using the pipette measure the weight of 40 mls of the filtered final solution. Record this weight; it will be used to calculate the SG of the final solution.
   XI. Use a known volume of H₂SO₄ acidified water, at the test pH, to wash any remaining solids in the vat onto the filter paper. This is also to wash the filter cake to remove any precipitates that may have formed. Ensure prior adding the H₂SO₄ that the cake has not cracked, as this will lead to incomplete washing. Record recovered volume.
   XII. Use a known volume of DI water to perform a final rinse of the filter cake. Record recovered volume, (ensure prior to adding H₂O that cake has not cracked, as this will lead to incomplete washing)
   XIII. Weigh dry empty vat and record weight
   XIV. All three solution samples are to be diluted and submitted for analysis
   XV. Place filter cake in low temperature oven to dry, record dry weight.
   XVI. The dry filter cake is to be bottle rolled to remove any lumps formed while drying and a ˜50 g sample to be split out of the residue and submitted for analysis. Ensure splitting of the sample is done accurately to guarantee a representative sample is submitted for analysis. The residues will be assayed for As, Sb, Ni, Co, Cu, Zn, Fe, Al, Ca, Mg, Mn, S (Total), S (Elemental), S2- (Sulphide), SO42-, C (Total), CO32- and an ICP Scan.

### Example 2

As described in Example 1 above, for the development of cultures capable of operating at high pH's, it was considered advantageous to select a starting culture that was not dependent on high ferric levels for leaching to progress. The Applicant has chalcopyrite and saline tolerant cultures that naturally operate in low ferric environments at conventional pH's (below 2), and the saline culture (Deposited as Accession No. V08/027581) was chosen as the starting point, as it also operates in low total iron environments due to tendency of the iron to precipitate in the highly saline environment.

As ferric iron begins to precipitate beyond pH 3 due to exceeding the solubility product, in a similar manner that precipitation is noted in the Applicant's saline culture, this culture was thought to be the best match to the proposed leaching conditions. Using the Applicant's procedures set out at Example 1 above, this culture was then adapted to operate at higher pH's.

A culture designated Ni—S-J065B was used in these studies, being derived directly from the culture prepared by the method of Example 1 above. The cultures were grown in 3 L stirred tank reactors and were maintained on nickel concentrate suspended in water. The desirable pH of the solution was achieved by addition of concentrated sulphuric acid. A customised water bath was used to regulate the temperature in the bioreactor at approximately 55° C. and aeration was provided to the culture by compressed air introduced into the mixing zone of the reactor.

Prior to its use, Ni—S-J065B was adapted to saline condition of 100 g/L Cl⁻ (115 g/L TDS), and a subculture of Ni—S-J065B was gradually adapted to a solution pH of 3.5. To distinguish the newly developed culture from the original inoculum, it was renamed to Ni—S-J069B.

### Sample Preparation of High Magnesia Ore

A high magnesia ore sample, designated J062A, was split using rotary splitter to provide samples as feed for bacterial development work and metallurgical analysis. A Head sample of the ore was assayed for Ni, Co, Zn, Cu, Fe, Al, Ca, Mg, Mn, S (Total), S (Elemental), S²⁻ (Sulphide), SO₄²⁻, C (Total), CO₃²⁻, and an ICP Scan.

Acid consumption studies were carried out on sample crushed to P₁₀₀=9.5 mm using a bottle roll technique. Concentrated sulphric acid was used to maintain the solution pH 1.8 to simulate conventional bioleaching conditions. Acid addition was recorded and used to calculate the total quantity of acid consumed by the ore.

### Results

### Head Grade and Mineralogical Characterisation

Head grade assay revealed the ore, J062A, to contain 0.68% nickel, 11.30% iron and 20.10% magnesium.

Mineralogical analysis indicates the main nickel bearing sulphide to be pentlandite at 2.18% of the total sample. Chalcopyrite is the other economic sulphide with a concentration of 0.14%. The main sulphide gangue occurs as pyrrhotite (2.18%). Magnesium is found in the form of forsterite (56.1%), edenite (18.8%), clinochlore (7.9%) and dolomite (2.9%). There appears to be a low level of liberation of nickel sulphides, however at the 3 mm crush size the majority of the grains have some exposure to the surface of the particle, indicating potential for leaching.

TABLE 1
Head grade of ore sample

| | Element | | |
|---|---|---|---|
| | Ni | Fe | Mg |
| Grades (%) | 0.68 | 11.30 | 20.10 |

### Acid Consumption Test Based Using Bottle Roll

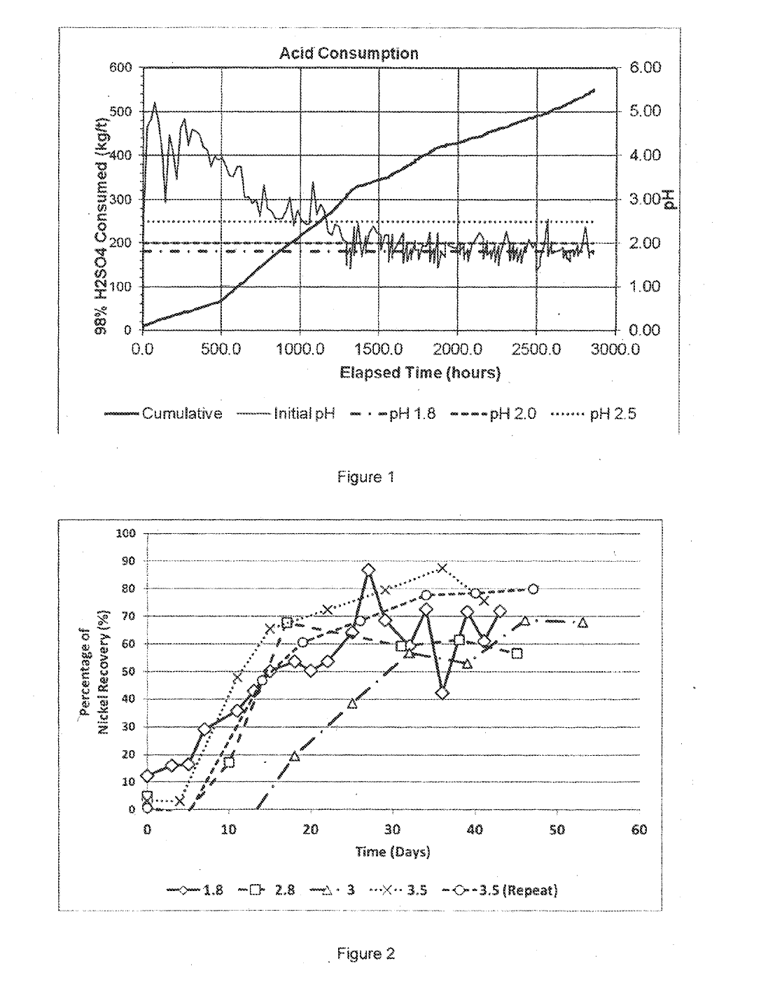

In order to understand the acid consumption characteristic of the ore under bioleaching solution acid consumption tests using a bottle roll technique at pH 1.8 was carried on ore crushed to 100% passing 9.5 mm. FIG. 1 shows that the solution pH took 54 days to reach a value of 1.8 and consumed ˜311 kg of 98% H₂SO₄ per tonne of ore. The ore sample continues to consume significant quantity of acid when the pH condition was maintained at value of 1.8. Upon termination of the study on day 119 (2856 hrs) the ore sample reported an acid consumption value of 558 kg t-1 ore.

### Bacterial Leaching Study

The bioleaching of J062A ore was carried out at a temperature of 55° C. at various solution pH value. Preliminary results indicated that nickel recoveries between all cultures were similar, see FIG. 2. Cultures leaching at pH of 3.5 were able to obtain a slightly higher nickel recovery in contrast to those operating at solution pH's less than 3.5 and comparable to the result obtained at pH 1.8. At solution pH's of 1.8 and 3.5, the cultures were able to leach more than 75% of the nickel in the ore. Cultures maintained at pH 2.8 and 3 obtained the least nickel recovery at approximately 56.7% and 67.9%, respectively.

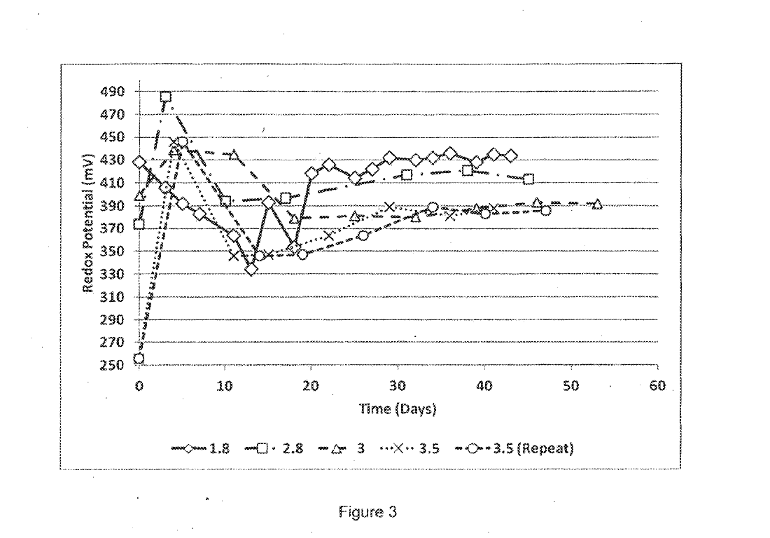

Redox potential measurements throughout the study revealed that the readings fluctuated during the first 20 days in all cultures before increasing and stablising at a constant value, as seen in FIG. 3. At the end of the study, cultures grown at pH 1.8 and 2.8 reported an ORP value of 434 mV and 413 mV vs Ag/AgCl, respectively. For cultures maintained at pH 3 and 3.5 redox potential was found to be in the range of 380 to 395 mV v's Ag/AgCl.

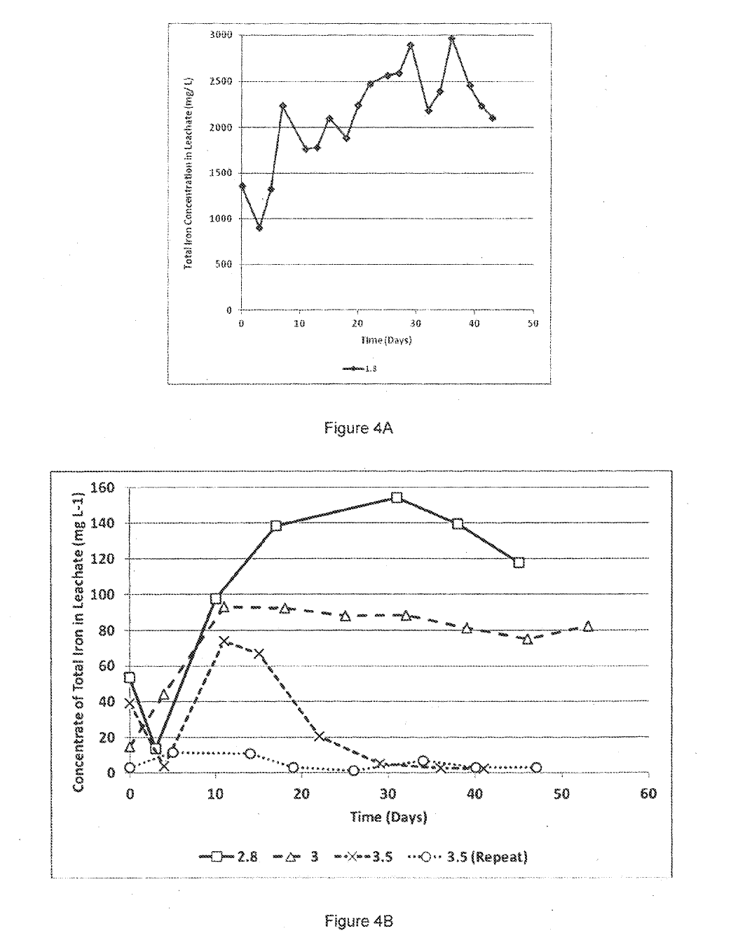

As is shown in FIGS. 4A and 4B, the concentration of total iron in solution monitored during the experiment clearly shows the effects of pH on the iron in solution. At end of the study the total amount of dissolved iron in solution for the culture grown in pH 1.8 solution was 2.10 g/L. The tests conducted at pH values of 2.8 and 3 showed a significant decrease of dissolved iron, with a final concentration of approximately 117 mg/L and 82 mg/L of total iron, respectively. Minimal iron was found to remain in solution for the culture grown at pH 3.5 (≦4 mg/L).

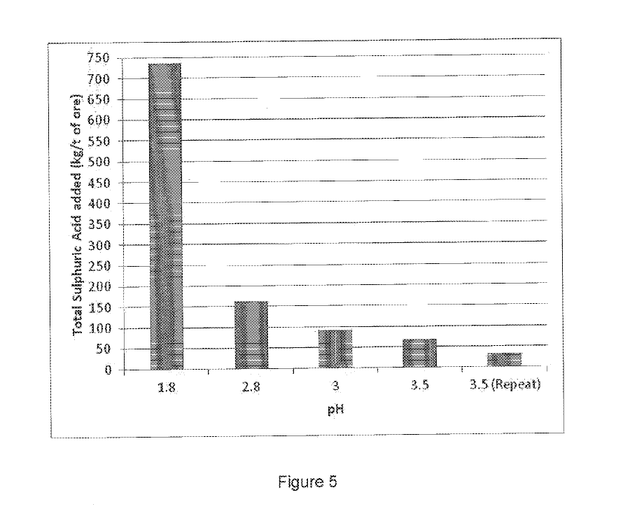

Sulphuric acid used to maintain the solution pH for each of the cultures was recorded during the study and used as a method of comparing acid consumptions between the tests. Preliminary analysis revealed that the cumulative quantity of acid needed to maintain the solution pH at low pH, is greater than for those tests at higher pH, as can be seen with reference to FIG. 5. The test at pH 1.8 recorded an acid consumption of 736 kg of acid per ton of ore. The quantity of acid required at pH 2.8 and 3 were 163 kg/t and 91 kg/t, respectively. At pH 3.5 acid addition was between 11 and 22 times less than those reported at pH 1.8.

It should be noted that acid consumptions are comparatively higher compared to the bottle roll test above due mainly to the much finer particle size of the leach test, compared with the bottle roll test.

As can be seen with reference to the above description, a microbial culture capable of leaching at high pH, and in saline solution of 100 g/L Cl⁻ was developed using the Applicant's procedures. The results have shown the culture is capable of leaching ore containing high levels of magnesium with solution pH values of 3 to 3.5 resulting in nickel recovery greater than 70%. Under these conditions, initial test work has indicated low levels of iron in solution and the acid consumption was reduced significantly, from approximately 736 kg/t of ore at pH 1.8 to 33 kg/t of ore at pH 3.5.

### Example 3

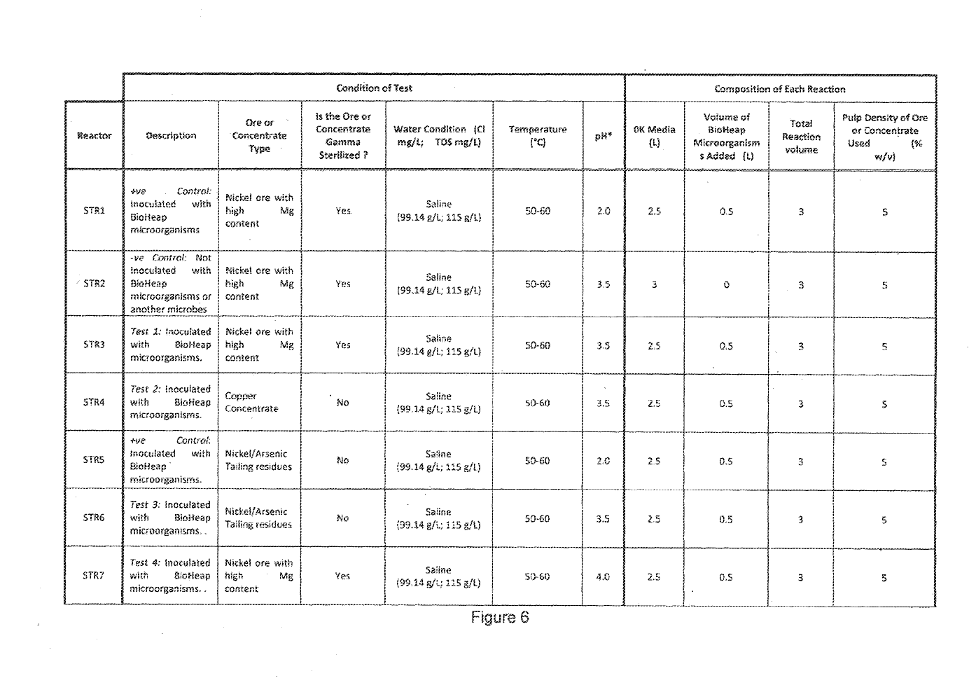

Further testing was undertaken, in the manner described for Example 2 above, to investigate the performance of the Applicant's bacterial culture. A summary of the controls and variables utilised in this testing is provided in the Table in FIG. 6. For each individual test in FIG. 6, the pH is maintained at a level as noted. In any event, where pH level starts to decrease as a result the bioleaching reaction and not of through the manual addition of acids, the pH level is left to decrease without adjustment through the addition of basic compounds.

The table immediately below shows the summary of head assay results on the ore and concentrate used in this study:

| | Percentage of Metal (%) | | | | | |
|---|---|---|---|---|---|---|
| Sample ID | Cu | Ni | Co | Mg | As | Fe |
| Nickel ore | N/A | 0.73 | 0.03 | 21.00 | N/A | 12.0 |
| with high Mg | | | | | | |
| content | | | | | | |
| Copper | 22.61 | N/A | N/A | 0.249 | N/A | 27.8 |
| Concentrate | | | | | | |
| Nickel/Arsenic | N/A | 8.60 | N/A | N/A | 1.56 | 37.7 |
| Tailing | | | | | | |

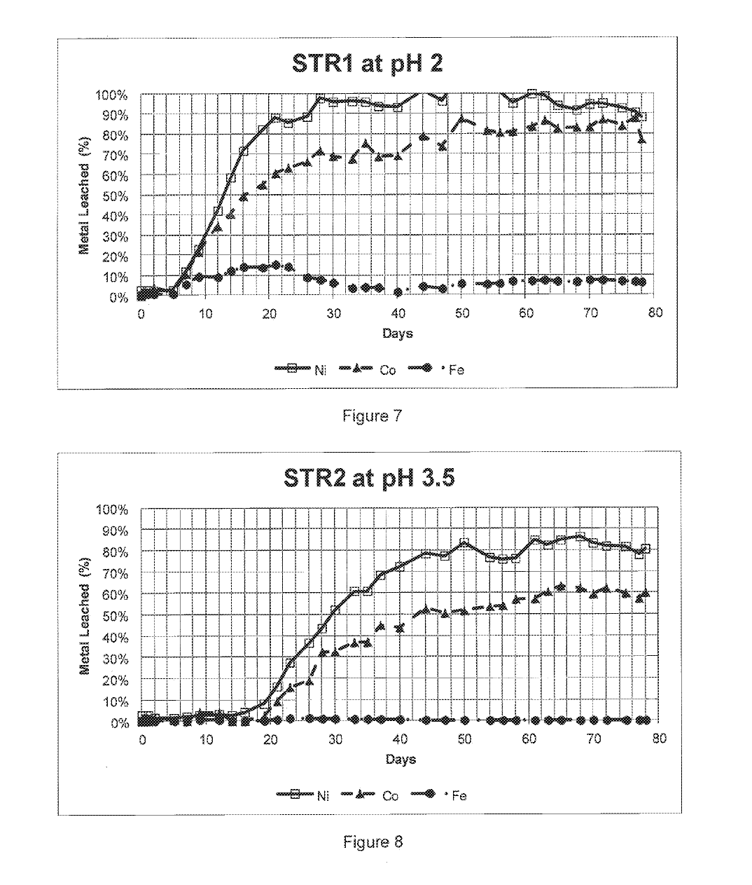

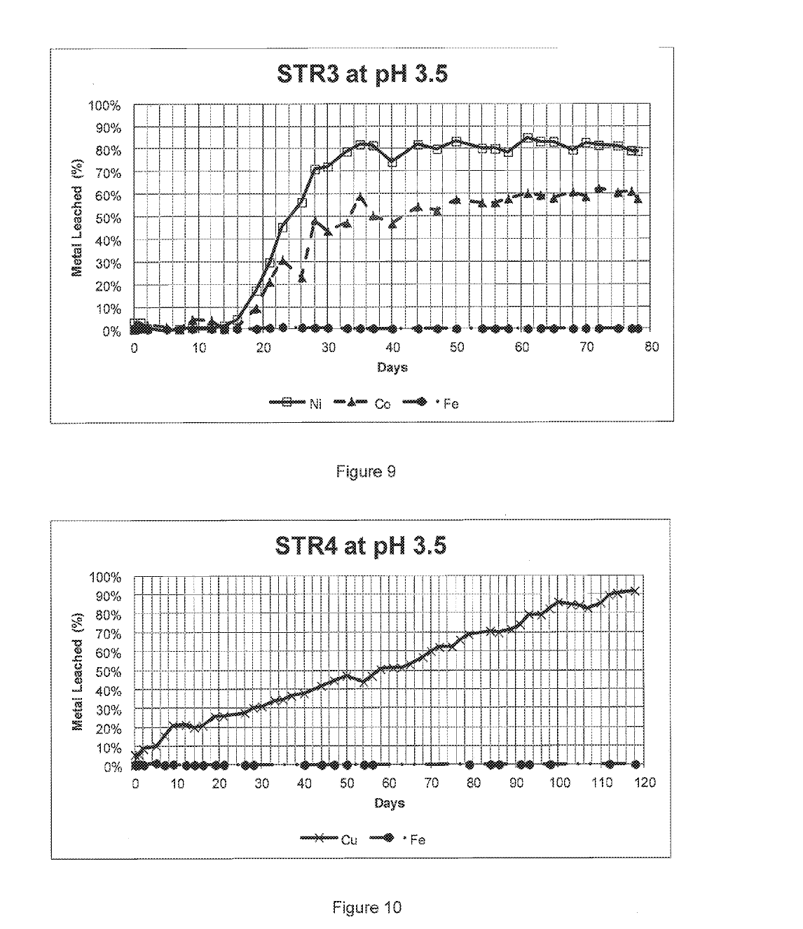

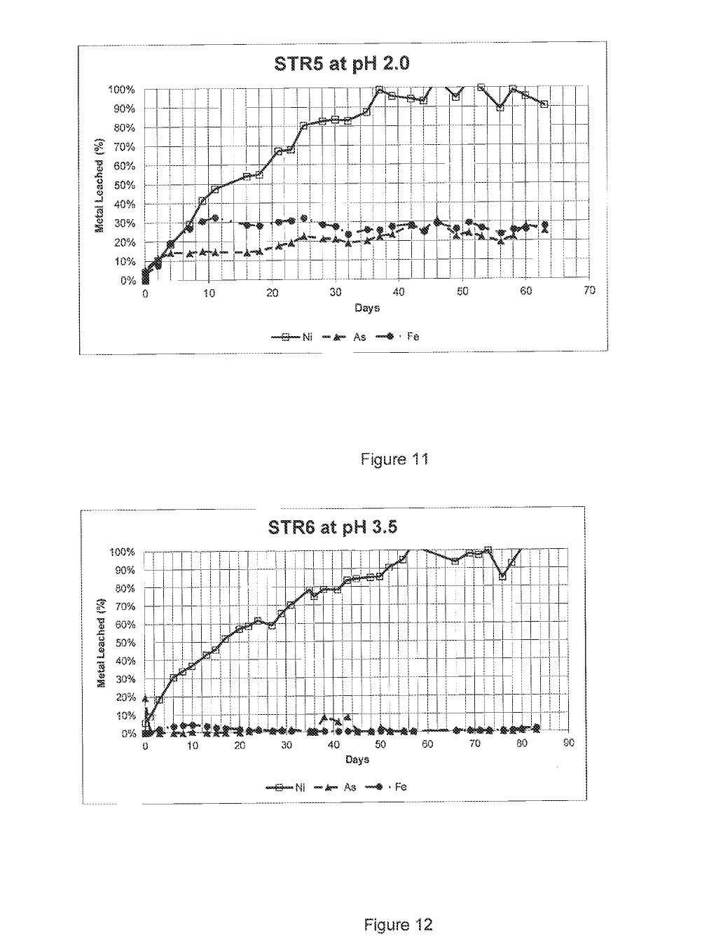

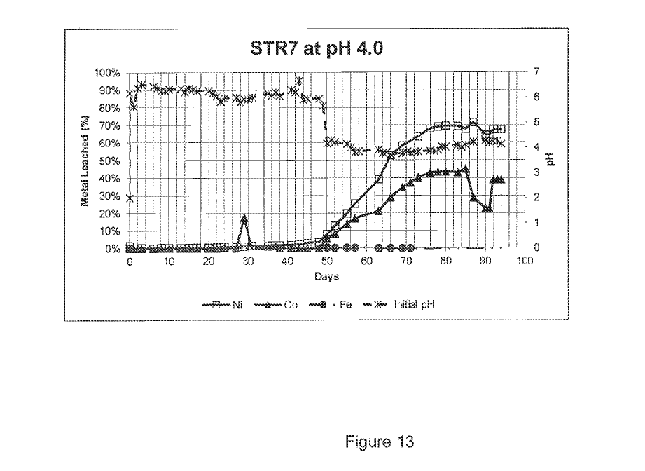

FIGS. 7 to 13 show the residue corrected percentage of metal leached from amenability study on each of STR1 to STR7, respectively, with nickel ore having high Mg content maintained at pH 3.5 at a pulp density of 5% w/v. It should be noted that the Applicant's cultures/microorganisms were not inoculated in this test. It should be noted that due to iron precipitation at pH greater than 3 residue correction was not conducted for iron. Rather, the liquor assay result is used.

The table immediately below provides a summary of metal recoveries based on residue assay and acid consumed for the completed test:

| | | | | | | | *Acid consumed |
|---|---|---|---|---|---|---|---|
| | | Metal of Recoveries based on Residue Assay (%) | | | | | (98% H₂SO₄ |
| Sample | Description | Ni | Co | Cu | As | Fe | kg/t test material) |
| STR1 | +ve Control at pH 2.0, | 94.06 | 83.78 | N/A | N/A | 14.46 | 408.13 |
| | Nickel Ore with | | | | | | |
| | high Mg content | | | | | | |
| STR2 | −ve Control at pH 3.5, | 83.02 | 60.06 | N/A | N/A | 7.69 | 82.13 |
| | Nickel Ore with | | | | | | |
| | high Mg content | | | | | | |
| STR3 | Test 1 at pH 3.5, | 81.57 | 59.98 | N/A | N/A | 6.80 | 85.33 |
| | Nickel Ore with | | | | | | |
| | high Mg content | | | | | | |
| STR4 | Test 2 pH 3.5, | N/A | N/A | 91.60 | N/A | 24.97 | 85.42 |
| | Copper | | | | | | |
| | Concentrate | | | | | | |
| STR5 | +ve Control pH 2.0, | 97.04% | N/A | N/A | 24.38% | 26.91 | 254.60 |
| | Nickel/Arsenic | | | | | | |
| | Tailing | | | | | | |
| STR6 | Test 3 pH 3.5, | 94.72 | N/A | N/A | 0.68 | 2.00 | 35.36 |
| | Nickel/Arsenic | | | | | | |
| | Tailing | | | | | | |
| #STR7 | Test 4 pH 4.0, | 67.50 | 38.97 | N/A | N/A | 0 | 84.00 |
| | Nickel Ore with | | | | | | |
| | high Mg content | | | | | | |

#STR7 is completed but due to delay in obtaining residues assay results only solution based recovery data is used.

The total acid consumption value noted in the table above is when nickel or copper reach a maximum percentage of recovery.

For each culture, initial pH and adjusted pH values were measured. The pH was adjusted by the addition of 98% H₂SO₄. Cumulative sulphuric acid is calculated in kilograms of 98% H₂SO₄ consumed per ton of ore. When acid is not added cumulative value reach steady state.

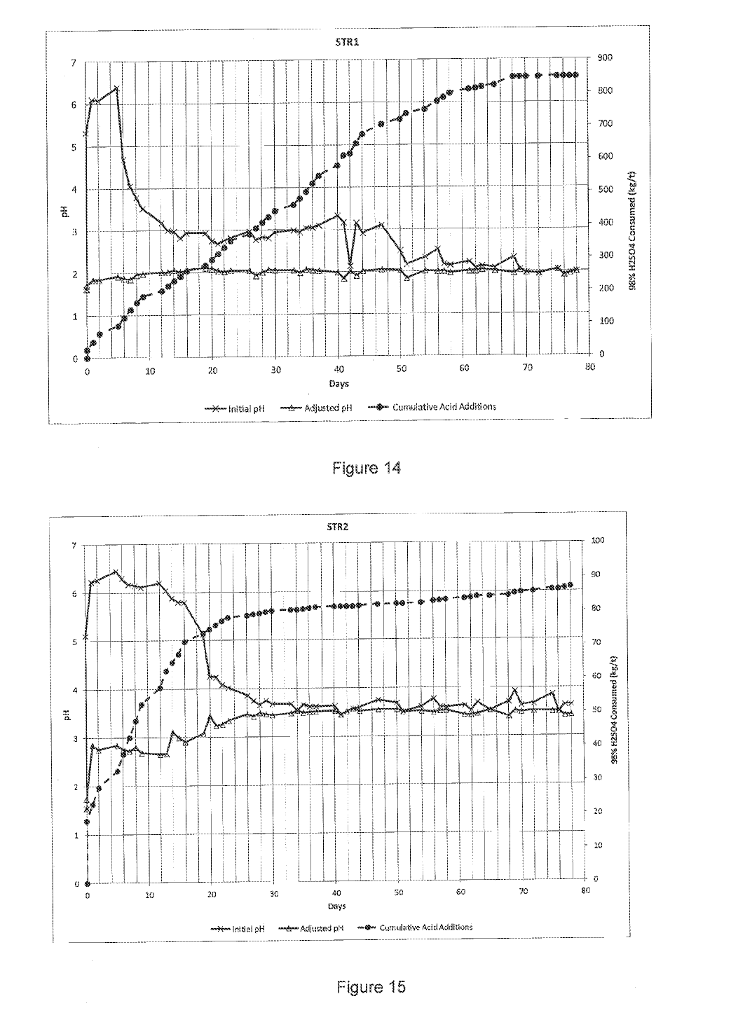

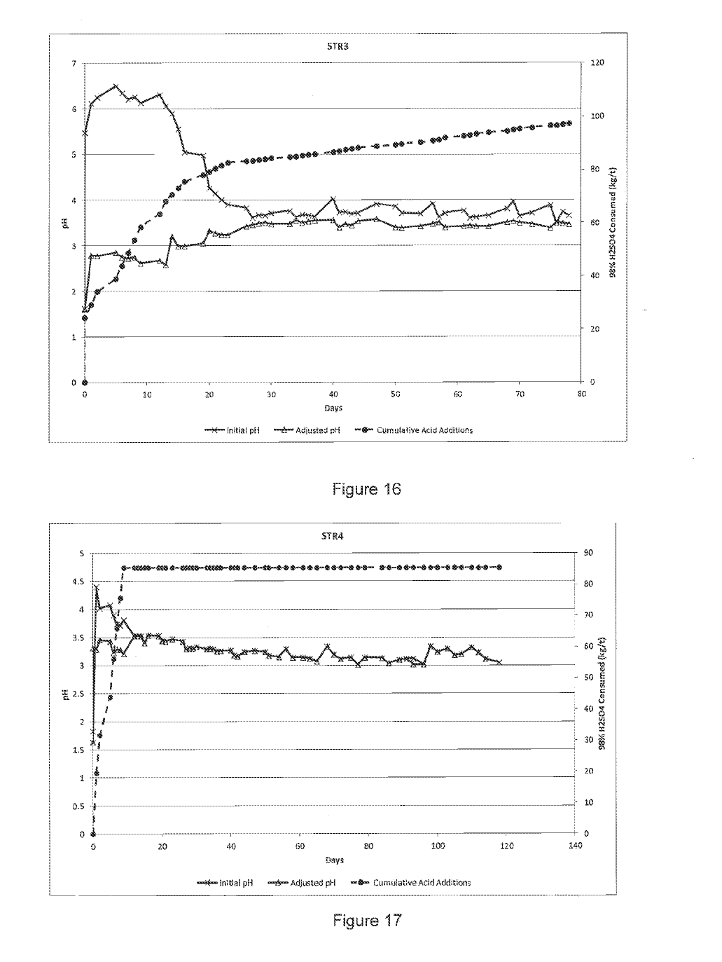

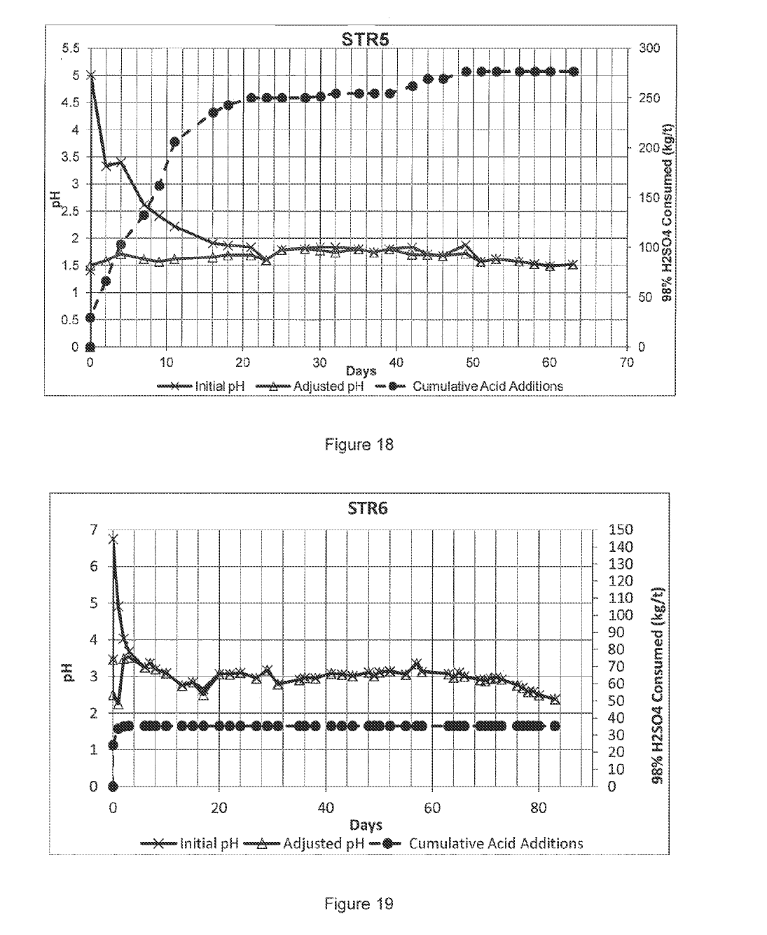

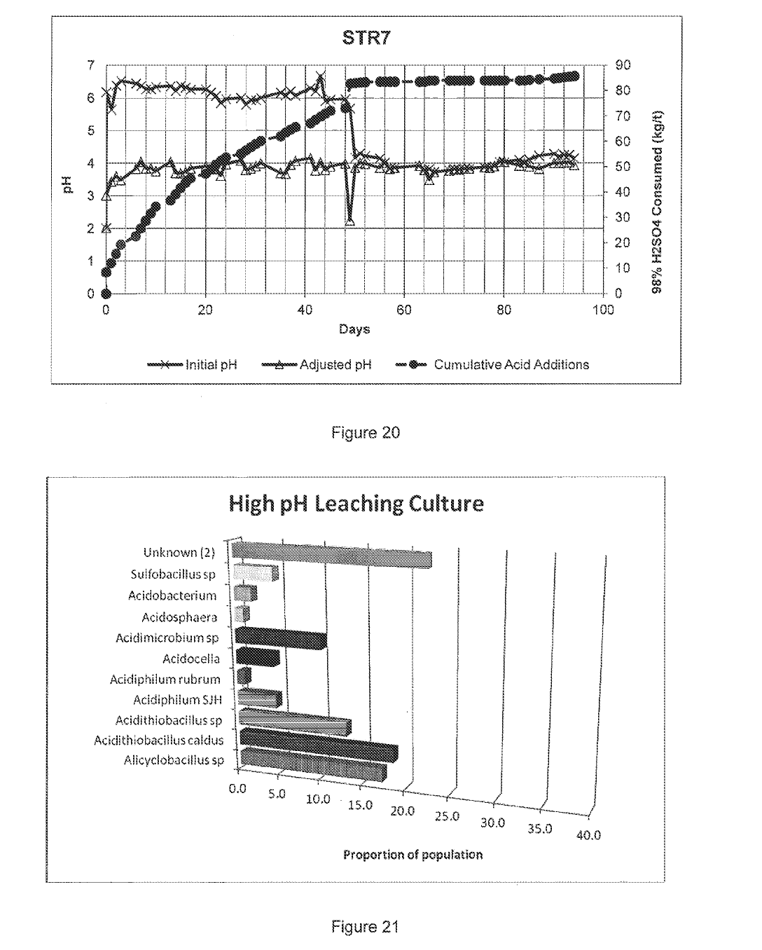

In FIGS. 14 to 20 there are shown plots of pH during testing for each culture. Initial pH and adjusted pH values were measured. The pH was adjusted by the addition of 98% H₂SO₄. Cumulative sulphuric acid is calculated in kilograms of 98% H₂SO₄ consumed per ton of ore. When acid is not added cumulative value reach steady state.

A sample of the Applicant's high pH leaching culture was submitted for microbial characterisation studies. Terminal restriction fragment length polymorphism (T-RFLP) technique was used to identify the microbes present in the culture. The majority of the microbes identified in the culture are Acidithiobacillus caldus, Alicyclobacillus sp, Acidithiobacillus sp and Acidimicrobium sp (see FIG. 21). Other minor microorganism presence are from the genera Acidobacterium, Acidosphaera, Acidocella and species Sulfobacillus sp, Acidiphilum rubrum and Acidiphilum SJH. In addition, archaea specie Acidianus brierleyi was also identified. It should be noted that the result obtained here does not suggest the culture consists of only these microbes. Other bacteria or archaea species may be presence in presently undetectable levels. It is also understood that population dynamics in the culture may change according to the changes of condition at various leaching phases. Surprisingly, a number of species identified have not been known to operate at highly saline conditions, such as 99.14 g/L Cl⁻, nor operate/survive at 50° C. or higher. Their presence demonstrates the success of the adaption process of the present invention.

Modifications and variations such as would be apparent to the skilled addressee are considered to fall within the scope of the present invention.

### REFERENCES

1. Cameron, R. A., Lastra, R., Mortazavi, S., Bedard, P. L., Morin, L., Gould, W. D., Kennedy, K. J., 2009a. Bioleaching of a low-grade ultramafic nickel sulphide ore in stirred-tank reactors at elevated pH. Hydrometallurgy 97, 213-220.
2. Cameron, R. A., Lastra, R., Mortazavi, S., Gould, W. D., Thibault, Y., Bedard, P. L., Morin, L., Kennedy, K. J., 2009b. Elevated-pH bioleaching of a low-grade ultramafic nickel sulphide ore in stirred-tank reactors at 5 to 45° C. Hydrometallurgy 99, 77-83.
3. Cameron, R. A., Yeung, C. W., Greer, C. W., Gould, W. D., Mortazavi, S., Bedard, P. L., Morin, L., Lortie, L., Dinardo, O., Kennedy, K. J., 2010. The bacterial community structure during bioleaching of a low-grade nickel sulphide ore in stirred-tank reactors at different combinations of temperature and pH. Hydrometallurgy 104, 207-215.
4. Dopson, M., Lövgren, L., Boström, D., 2009. Silicate mineral dissolution in the presence of acidophilic microorganisms: Implications for heap bioleaching. Hydormetallurgy 96, 288-293.
5. Halien, A. -k., Rahunen, N., Kaksonen, A., Puhakka, J. A., 2009. Heap bioleaching of a complex sulfide ore: Part I. Effect of pH on metal extraction and microbial composition in pH controlled columns. Hydormetallurgy 98, 92-100.
6. Li, H., Ke, J., 2001a. Influence of Ni2+ and Mg2+ on the growth and activity of Cu2+-adapted thiobacillus ferrooxidans. Hydrometallurgy 61, 151-156.
7. Li, H., Ke, J., 2001 b. Influence of Cu2+ and Mg2+ on the growth and activity of Ni2+-adapted thiobacillus ferrooxidans. Minerals Engineering 14, 113-116.
8. Plumb, J. J., Muddle, R., Franzmann, P. D., 2008. Effect of pH on rates of iron and sulfur oxidation by bioleaching organisms. Minerals Engineering 21, 76-82.
9. Qin, W., Zhen, S., Yan, Z., Campbell, M., Wang, J., Wang, J., Liu, K., Zhang, Y., 2009. Heap bioleaching of a low-grade nickel-bearing sulfide ore containing high levels of magnesium as olivine, chlorite and antigorite. Hydormetallurgy 98, 58-65.
10. Rawlings, D. E., Dew, D., du Plessis, C., 2003. Biomineralization of metal-containing ores and concentrates. Trends Biotechnology 21, 38-44.
11. Rohwerder, T., Gehrke, T., Kinzler, K., Sand, W., 2003 Bioleaching review part A: Progress in bioleaching: fundamentals and mechanisms of bacterial metal sulfide oxidation. Applied Microbiology and Biotechnology 63, 239-248.
12. Ströberg, B., Banwart, S. A., 1999. Expermental study of acidity-consuming processes in mining waste rock: some influences of mineraology and particle size. Appl. Geochem. 14, 1-16.
13. Watling, H. R., 2006. The bioleaching of sulphide minerals with emphasis on copper sulphides. A review. Hydrometallurgy. 84, 81-108.
14. Zhen, S., Yan, Z., Zhang, Y., Wang, J., Campbell, M., Qin, W., 2009. Column bioleaching of a low grade nickel-bearing sulfide ore containing high magnesium as olivine, chlorite and antigorite. Hydrometallurgy. 96, 337-341.

1. A method of adaptation of a bacterial culture for use in the treatment of ores and concentrates, the method comprising the method steps of:
Exposing the bacterial culture to increasing levels of pH over a period of time,
whereby the bacterial culture is operative at a pH of above 2.5.

2. A method according to claim 1, wherein the bacterial culture is operative at a pH of above 3.5.

3. A method according to claim 1 or 2, wherein the bacterial culture is operative at a pH of between about 3.5 to 5.

4. A method according to claim 1, wherein the bacterial culture is a salt tolerant culture.

5. A method according to claim 4, wherein the salt tolerant culture is capable of operation at chloride concentrations of greater than about 100 g/L.

6. A method according to claim 4, wherein the salt tolerant culture is capable of operation at total dissolved solids (TDS) levels of greater than about 115 g/L.

7. A method according to claim 1, wherein the temperature of step (i) is about 45 to 55° C.

8. A method according to claim 1, wherein the temperature of step (i) is between about 50 to 55° C.

9. A method according to claim 1, wherein the bacterial culture is a bacterial culture capable of oxidising sulphide ores and concentrates.

10. A leaching process comprising the process steps of:
(i) Adapting a bacterial culture to operate at a pH of above 2.5; and
(ii) Leaching an ore or concentrate to which the adapted bacterial culture of step (i) has been added or is added at a pH above 2.5.

11. A leaching process according to claim 10, wherein the adaptation of the bacterial culture of step (i) adapts that bacterial culture to operate at a pH above 3.5.

12. A leaching process according to claim 10 wherein the adaptation of the bacterial culture of step (i) adapts that bacterial culture to operate at a pH between about 3.5 to 5.

13. A leaching process according to claim 10, wherein the adaptation of the bacterial culture of step (i) adapts that bacterial culture to operate at a pH of about 5.

14. A leaching process according to claim 10, wherein the leaching of step (ii) is conducted at the same or similar pH to that to which the bacterial culture has been adapted in step (i).

15. A leaching process according to claim 10, wherein the bacterial culture may be adapted in step (i) to additional conditions, including conditions of salinity.

16. A leaching process according to claim 10, wherein the leaching of step (ii) is conducted at the same or similar additional conditions to that to which the bacterial culture has been adapted in step (i).

17. A leaching process according to claim 10, wherein the leach is conducted in the form of a heap leach utilising one or more heaps.

18. A leaching process according to claim 10, wherein the leach is conducted in one or more tanks.

19. A leaching process according to claim 10, wherein the leach provides a target metal recovery of greater than about 70% at a pH of between about 3.0 to 3.5.

20. A leaching process according to claim 10, wherein the leach provides a target metal recovery of greater than 70% at a pH of greater than about 3.5.

21. A leaching process according to claim 10, wherein the target metal is nickel.

22. A leaching process according to claim 10, wherein the leaching process consumes relatively low amounts of acid.

23. A leaching process according to claim 10, wherein the leaching process produces a pregnant leach solution containing relatively low levels of iron.
